(19) United States
(12) Patent Application Publication
(10) Pub. No.: US 2016/0206873 A1
(43) Pub. Date: Jul. 21, 2016

(54) METHODS AND SYSTEMS FOR NEUROSTIMULATION AT THE TRACT OF LISSAUER

(71) Applicant: PACESETTER, INC., Sylmar, CA (US)

(72) Inventors: Wenbo Hou, Santa Clarita, CA (US); Melanie Goodman Keiser, McKinney, TX (US)

(21) Appl. No.: 14/597,924

(22) Filed: Jan. 15, 2015

(51) Int. Cl. A61N 1/05; A61N 1/36

(52) U.S. Cl. CPC A61N 1/0553; A61N 1/36071; A61N 1/36139

(57) ABSTRACT

The present disclosure provides methods and systems for neurostimulation at the tract of Lissauer. A neurostimulation system includes an implantable pulse generator, and a lead assembly electrically coupled to the implantable pulse generator, the lead assembly including a plurality of sensing electrodes configured to sense pain signals at a tract of Lissauer of a subject, and a plurality of stimulating electrodes configured to apply stimulation to a dorsal column of the subject based on the sensed pain signals.

### A. FIELD OF THE DISCLOSURE

The present disclosure relates generally to neurostimulation systems, and more particularly to closed-loop neurostimulation systems for use at the tract of Lissauer.

### B. BACKGROUND ART

Neurostimulation is a treatment method utilized for managing the disabilities associated with pain, movement disorders such as Parkinson's Disease (PD), dystonia, and essential tremor, and also a number of psychological disorders such as depression, mood, anxiety, addiction, and obsessive compulsive disorders.

At least some known neurostimulation systems are closed-loop spinal cord stimulation (SCS) systems based on neurological sensing systems. In one system, for example, for multiple patients, octopolar electrodes were implanted at the anatomical midline of the epidural space in the spinal cord as verified by fluoroscope. Further, evoked compound action potential (ECAP) neurological signals sensed from Aβ fibers have been approved for incorporation into an implantable pulse generator (IPG) device for closed SCS in which the ECAPs are sensed for a patient's positional changes.

### BRIEF SUMMARY OF THE DISCLOSURE

In one embodiment, the present disclosure is directed to a neurostimulation system. The neurostimulation system includes an implantable pulse generator, and a lead assembly electrically coupled to the implantable pulse generator, the lead assembly including a plurality of sensing electrodes configured to sense pain signals at a tract of Lissauer of a subject, and a plurality of stimulating electrodes configured to apply stimulation to the dorsal column of the subject based on the sensed pain signals.

In another embodiment, the present disclosure is directed to a lead assembly for use in a neurostimulation system. The lead assembly includes a plurality of sensing electrodes configured to sense pain signals at a tract of Lissauer of a subject, and a plurality of stimulating electrodes configured to apply stimulation to the dorsal column of the subject based on the sensed pain signals.

In another embodiment, the present disclosure is directed to a method of operating a neurostimulation system including a plurality of sensing electrodes and a plurality of stimulating electrodes. The method includes implanting the neurostimulation system in a subject, sensing, using the plurality of sensing electrodes, pain signals at a tract of Lissauer of the subject, and applying, using the plurality of stimulating electrodes, stimulation to the dorsal column of the subject based on the sensed pain signals.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIGS. 2A-2C are schematic views of stimulation portions that may be used with the stimulation system of FIG. 1.

FIG. 3 is a schematic diagram of the tract of Lissauer.

FIG. 4 is a schematic diagram of one embodiment of a lead assembly that may be used with the stimulation system of FIG. 1.

FIG. 5 is a schematic diagram of the lead assembly of FIG. 4 implanted in a subject.

FIG. 6 is a schematic diagram of an alternative lead assembly that may be used with the stimulation system of FIG. 1.

FIGS. 7A and 7B are schematic diagrams of the lead assembly of FIG. 6 implanted in a subject.

FIG. 8 is a schematic diagram of an alternative lead assembly that may be used with the stimulation system of FIG. 1.

FIG. 9 is a schematic diagram of the lead assembly of FIG. 8 implanted in a subject.

Corresponding reference characters indicate corresponding parts throughout the several views of the drawings.

### DETAILED DESCRIPTION OF THE DISCLOSURE

The present disclosure provides methods and systems for neurostimulation at the tract of Lissauer. A neurostimulation system includes an implantable pulse generator, and a lead assembly electrically coupled to the implantable pulse generator, the lead assembly including a plurality of sensing electrodes configured to sense pain signals at a tract of Lissauer of a subject, and a plurality of stimulating electrodes configured to apply stimulation to a dorsal column of the subject based on the sensed pain signals.

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. In SCS, electrical pulses are delivered to nerve tissue of the spinal cord for the purpose of chronic pain control. While a precise understanding of the interaction between the applied electrical energy and the nervous tissue is not fully appreciated, it is known that application of an electrical field to spinal nervous tissue can effectively inhibit certain types of pain transmitted from regions of the body associated with the stimulated nerve tissue to the brain. Specifically, applying electrical energy to the spinal cord associated with regions of the body afflicted with chronic pain can induce “paresthesia” (a subjective sensation of numbness or tingling) in the afflicted bodily regions.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

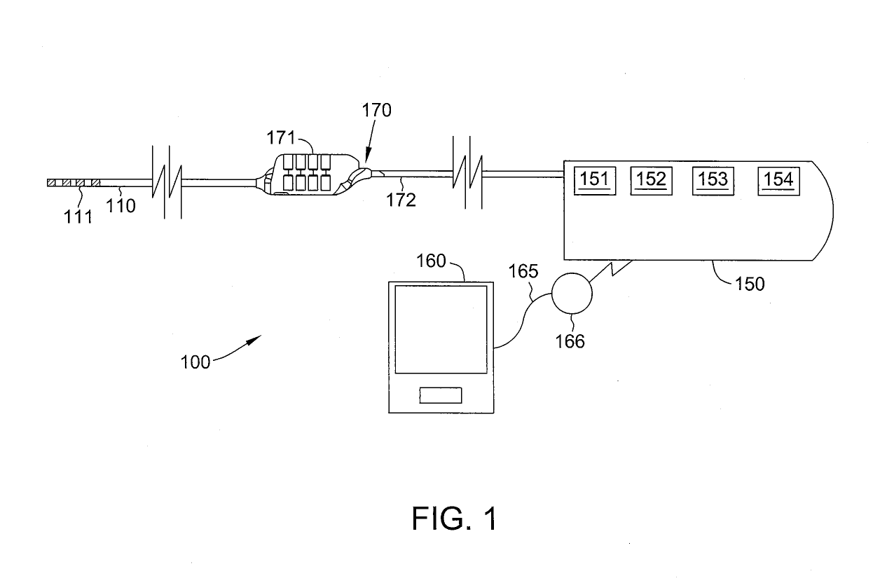

Referring now to the drawings and in particular to FIG. 1, a stimulation system is indicated generally at 100. Stimulation system 100 generates electrical pulses for application to tissue of a patient, or subject, according to one embodiment. System 100 includes an implantable pulse generator (IPG) 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of pulse generator 150 for execution by the microcontroller or processor to control the various components of the device.

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

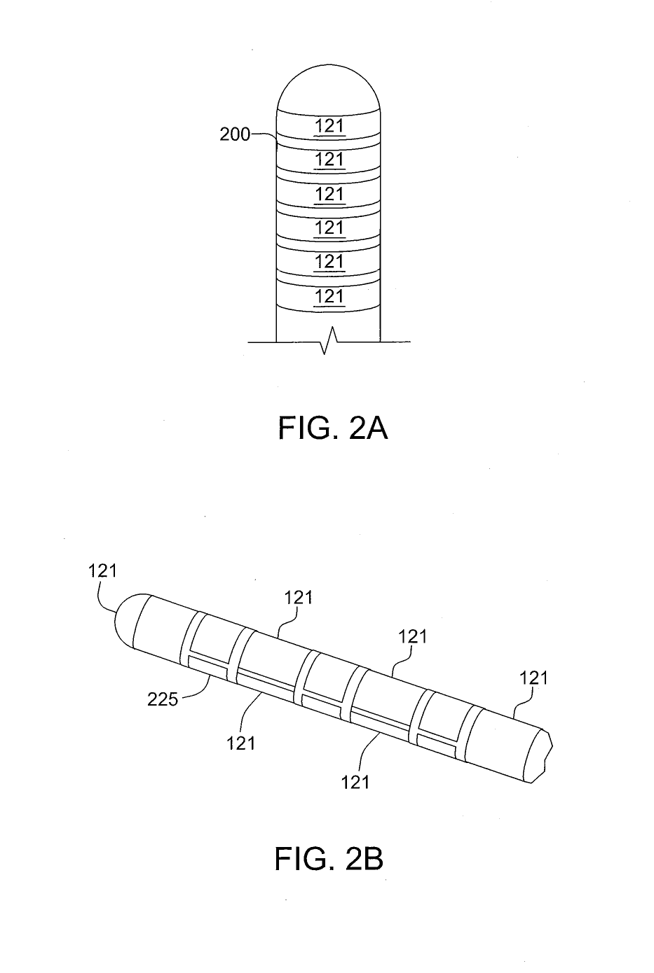

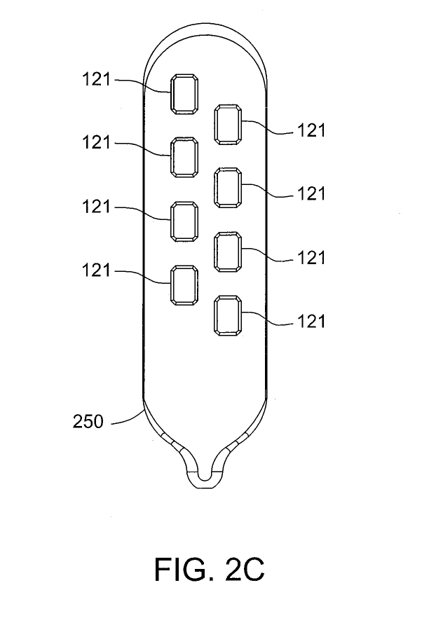

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2011/0072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Controller device 160 may be implemented to recharge battery 153 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of generator 150. The charging circuitry may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical, Inc. (Plano, Tex.). Example commercially available stimulation leads include the QUATTRODE™, OCTRODE™, AXXESS™, LAMITRODE™, TRIPOLE™, EXCLAIM™, and PENTA™ stimulation leads from St. Jude Medical, Inc.

The systems and methods described herein provide a closed-loop SCS by stimulating the dorsal column and sensing from the tract of Lissauer. Sensing leads measure action potentials from C or A-delta fibers in the tract of Lissauer within the spinal cord. A closed-loop system includes an IPG device, such as pulse generator 150 (shown in FIG. 1), with sensing and stimulating functions. In some embodiments, the system further includes at least one paddle lead with electrodes for stimulating purposes, and at least one lead for sensing neural signals from the tract of Lissauer.

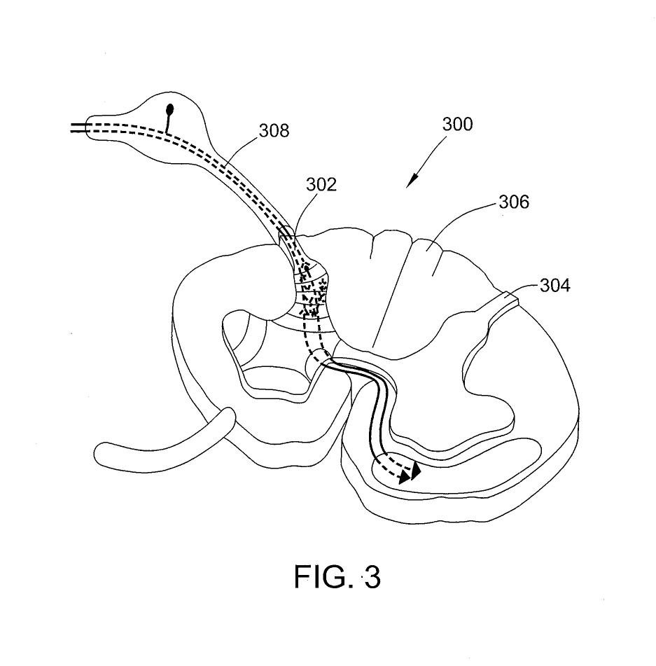

FIG. 3 is a schematic diagram of the tract of Lissauer 300. The tract of Lissauer 300 includes a right tract 302 and a left tract 304, with the dorsal column 306 positioned between right and left tracts 302 and 304. Peripheral nociceptive fibers 308 enter the dorsal horn and ascend the spinal cord in one or two segments to form the tract of Lissauer 300 before synapsing onto second-order neurons. The tract of Lissauer 300 can be found laterally, just above lamina l of the dorsal horn and proximate the entrance of the dorsal roots into the spinal cord. Pain signals, such as action potentials, can be sensed from the tract of Lissauer 300, as described in detail herein.

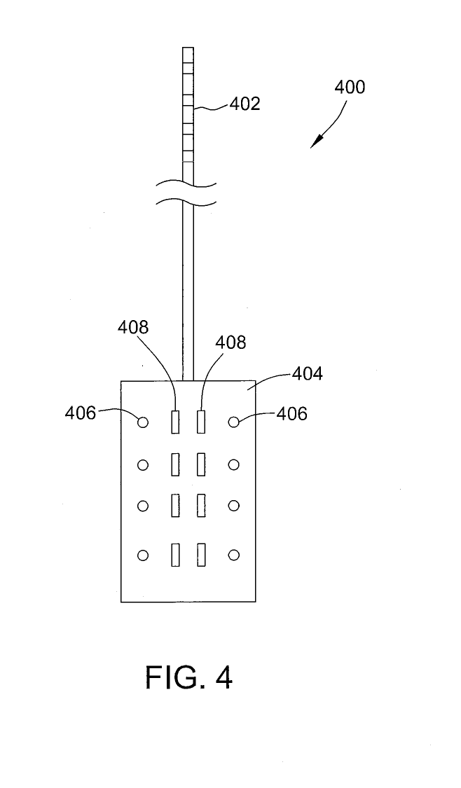

FIG. 4 is a schematic diagram of one embodiment of a lead assembly 400 that may be used to provide electrical stimulation and sensing at the tract of Lissauer 300. Lead assembly 400 includes a connector 402 (e.g., for connecting to an IPG) and a paddle lead 404. As shown in FIG. 4, paddle lead 404 includes a plurality of sensing electrodes 406 and a plurality of stimulating electrodes 408 arranged in a grid. In this embodiment, paddle lead 404 includes sixteen total electrodes, with two outer columns having four sensing electrodes 406 each, and two inner columns having four stimulating electrodes 408 each. Alternatively, paddle lead 404 may have any number and/or configuration of electrodes that enables lead assembly 400 to function as described herein.

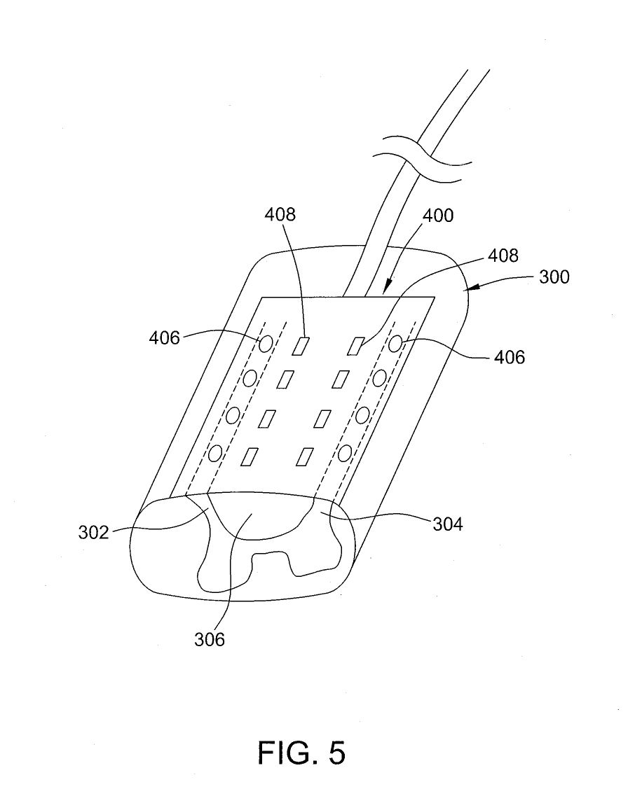

FIG. 5 is a schematic diagram of lead assembly 400 implanted in a subject. Lead assembly 400 is implanted such that stimulating electrodes 408 are positioned to stimulate dorsal column 306 for therapy purposes, and sensing electrodes 406 are positioned to sense pain signals in right and left tracts 302 and 304 without any motion effects. Further, the arrangement of sensing electrodes 406 facilitates sensing at different locations within right and left tracts 302 and 304.

To implant lead assembly 400, a laminectomy procedure may be used. In some embodiments, the implantation procedure may require removing bone laterally to expose and access the dorsal column and tract of Lissauer 300. After implantation, lead assembly 400 is activated for therapy. In operation, a series of pain signals are sensed by sensing electrodes 406 under different stimulation pulse configurations applied by stimulating electrodes 408. The stimulation pulse configuration that generates a minimal frequency of action potentials will generally be the optimal stimulation pulse configuration for ambulatory pain therapy. Accordingly, action potentials sensed by sensing electrodes 406 are used to control operation of stimulating electrodes 408, forming a closed-loop system.

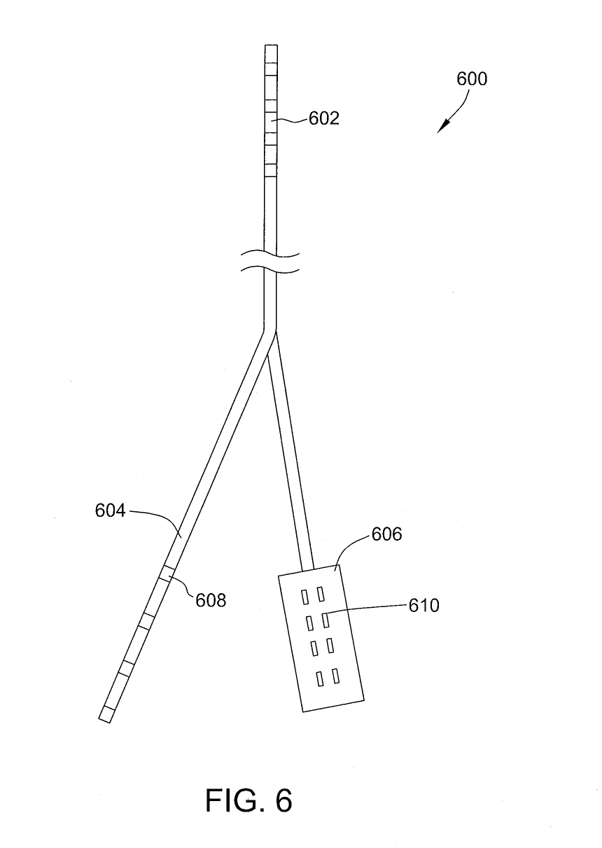

FIG. 6 is a schematic diagram of an alternative lead assembly 600 which has separate stimulating and sensing leads. Lead assembly 600 includes a connector 602 (e.g., for connecting to an IPG), a sensing lead 604, and a stimulating lead 606. In this embodiment, sensing lead 604 is a cylindrical lead including a plurality of sensing electrodes 608, and stimulating lead 606 is a paddle lead including a plurality of stimulating electrodes 610. Although FIG. 6 depicts four sensing electrodes 608 and eight stimulating electrodes 610, sensing lead 604 and stimulating lead 606 may include any number and/or configuration of electrodes that enables lead assembly 600 to function as described herein.

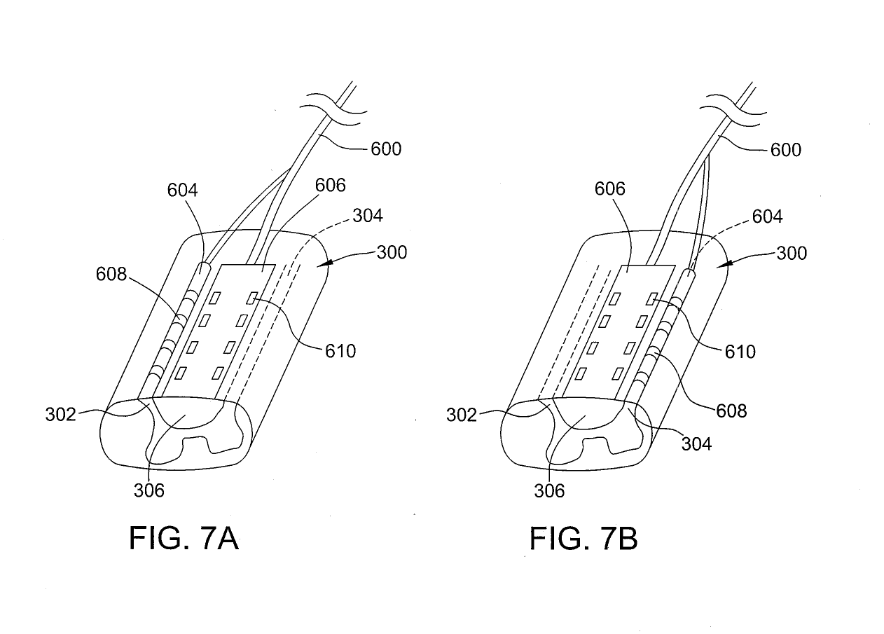

FIGS. 7A and 7B are schematic diagrams of lead assembly 600 implanted in a subject. Similar to lead assembly 400, lead assembly 600 may be implanted using a laminectomy procedure and operates similar to lead assembly 400. In FIG. 7A, sensing lead 604 is positioned on right tract 302, and in FIG. 7B, sensing lead 604 is positioned on left tract 304. Specifically, in this embodiment, sensing lead 604 is positioned ipsilaterally according to the pain location. If pain is located on the right side of the subject, sensing lead 604 is positioned on right tract 302. If pain is located on the left side of the subject, sensing lead 604 is positioned on left tract 304. If pain is centralized to the midline of the subject's body, or affects both sides of the body, sensing leads may be positioned on both right and left tracts 304 (similar to the configuration shown in FIG. 5).

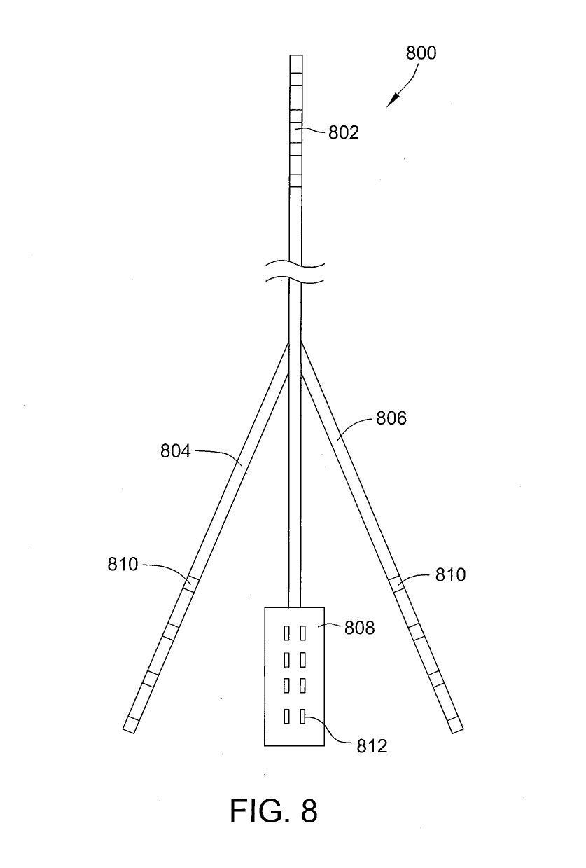

FIG. 8 is a schematic diagram of an alternative lead assembly 800. Lead assembly 800 includes a connector 802 (e.g., for connecting to an IPG), a first sensing lead 804, a second sensing lead 806, and a stimulating lead 808 arranged in a wedge-shaped configuration. In this embodiment, first and second sensing leads 804 and 806 are cylindrical leads including a plurality of sensing electrodes 810, and stimulating lead 808 is a paddle lead including a plurality of stimulating electrodes 812. Although FIG. 8 depicts four sensing electrodes 810 on each of first and second sensing leads 804, and eight stimulating electrodes 812, leads 804, 806, and 808 may include any number and/or configuration of electrodes that enables lead assembly 800 to function as described herein.

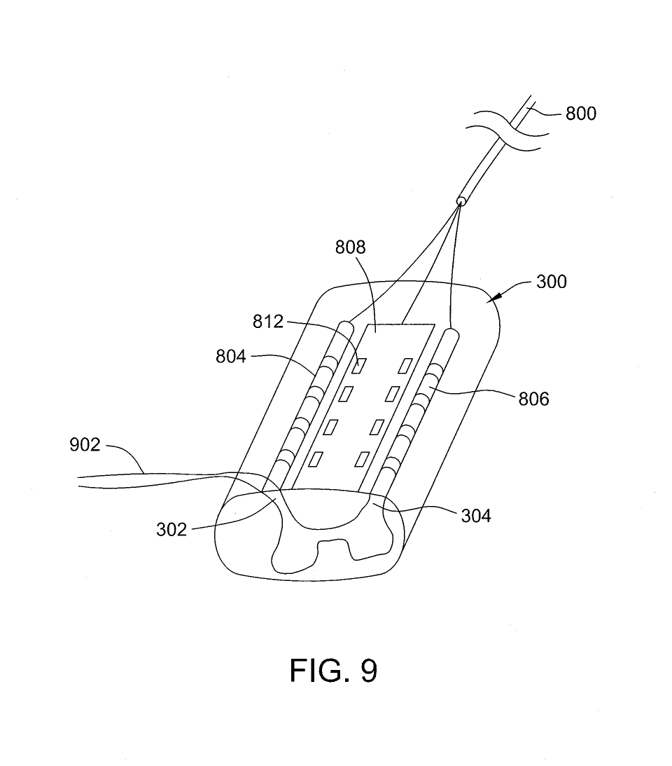

FIG. 9 is a schematic diagram of lead assembly 800 implanted in a subject. Similar to lead assembly 400, lead assembly 800 may be implanted using a laminectomy procedure and operates similar to lead assembly 400. As shown in FIG. 9, first sensing lead 804 is positioned on right tract 302, and second sensing lead 806 is positioned on left tract 304. This configuration may be desirable if pain is centralized to the midline of the subject's body, or affects both sides of the body. FIG. 9 also depicts the dorsal root 902 entering right tract 302. As compared to lead assembly 400, lead assembly 800 allows more flexibility when positioning sensing electrodes 810.

The lead assemblies described herein facilitate closed-loop neurostimulation at the tract of Lissauer. In embodiments where both the left and right tracts include sensing electrodes (see, e.g., FIGS. 5 and 9), an operator may select (e.g., by programming the IPG) which sensing electrodes are active. That is, although sensing electrodes are positioned on both the left and right tracts, an operator may elect to sense on only one side. Further, in the event of lead migration, electrodes on the lead assemblies may be reconfigured accordingly. For example, if the location of lead assembly 400 shown in FIG. 5 shifts such that at least some of stimulating electrodes 408 are positioned on the right or left tracts, those stimulating electrodes 408 can be reprogrammed to function as sensing electrodes. Lead migration may be detected, for example, by monitoring action potentials seen by the sensing electrodes.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A neurostimulation system comprising:
an implantable pulse generator; and
a lead assembly electrically coupled to the implantable pulse generator, the lead assembly comprising:
a plurality of sensing electrodes configured to sense pain signals at a tract of Lissauer of a subject; and
a plurality of stimulating electrodes configured to apply stimulation to a dorsal column of the subject based on the sensed pain signals.

2. The neurostimulation system of claim 1, wherein the lead assembly further comprises a paddle lead, and wherein the plurality of sensing electrodes and the plurality of stimulating electrodes are positioned on the paddle lead.

3. The neurostimulation system of claim 1, wherein the plurality of sensing electrodes and the plurality of stimulating electrodes are arranged on the paddle lead in a grid formation.

4. The neurostimulation system of claim 1, wherein the lead assembly further comprises:
a paddle lead, wherein the plurality of stimulating electrodes are positioned on the paddle lead; and
a cylindrical lead, wherein the plurality of sensing electrodes are positioned on the cylindrical lead.

5. The neurostimulation system of claim 4, wherein the cylindrical lead is configured to be positioned on one of a right tract of the tract of Lissauer and a left tract of the tract of Lissauer.

6. The neurostimulation system of claim 1, wherein the lead assembly further comprises:
a paddle lead, wherein the plurality of stimulating electrodes are positioned on the paddle lead;
a first cylindrical lead, wherein a first subset of the plurality of sensing electrodes are positioned on the first cylindrical lead; and
a second cylindrical lead, wherein a second subset of the plurality of sensing electrodes are positioned on the second cylindrical lead.

7. The neurostimulation system of claim 6, wherein the first cylindrical lead is configured to be positioned on a right tract of the tract of Lissauer, and wherein the second cylindrical lead is configured to be positioned on a left tract of the tract of Lissauer.

8. A lead assembly for use in a neurostimulation system, the lead assembly comprising:
a plurality of sensing electrodes configured to sense pain signals at a tract of Lissauer of a subject; and
a plurality of stimulating electrodes configured to apply stimulation to a dorsal column of the subject based on the sensed pain signals.

9. The lead assembly of claim 8, further comprising a paddle lead, wherein the plurality of sensing electrodes and the plurality of stimulating electrodes are positioned on the paddle lead.

10. The lead assembly of claim 9, wherein the plurality of sensing electrodes and the plurality of stimulating electrodes are arranged on the paddle lead in a grid formation.

11. The lead assembly of claim 8, further comprising:
a paddle lead, wherein the plurality of stimulating electrodes are positioned on the paddle lead; and
a cylindrical lead, wherein the plurality of sensing electrodes are positioned on the cylindrical lead.

12. The lead assembly of claim 11, wherein the cylindrical lead is configured to be positioned on one of a right tract of the tract of Lissauer and a left tract of the tract of Lissauer.

13. The lead assembly of claim 8, further comprising:
a paddle lead, wherein the plurality of stimulating electrodes are positioned on the paddle lead;
a first cylindrical lead, wherein a first subset of the plurality of sensing electrodes are positioned on the first cylindrical lead; and
a second cylindrical lead, wherein a second subset of the plurality of sensing electrodes are positioned on the second cylindrical lead.

14. The lead assembly of claim 13, wherein the first cylindrical lead is configured to be positioned on a right tract of the tract of Lissauer, and wherein the second cylindrical lead is configured to be positioned on a left tract of the tract of Lissauer.

15. A method of operating a neurostimulation system including a plurality of sensing electrodes and a plurality of stimulating electrodes, the method comprising:
implanting the neurostimulation system in a subject;
sensing, using the plurality of sensing electrodes, pain signals at a tract of Lissauer of the subject; and
applying, using the plurality of stimulating electrodes, stimulation to a dorsal column of the subject based on the sensed pain signals.

16. The method of claim 15, wherein implanting the neurostimulation system comprises implanting the neurostimulation system using a laminectomy procedure.

17. The method of claim 15, wherein implanting the neurostimulation system comprises implanting a neurostimulation system including a paddle lead, and wherein the plurality of sensing electrodes and the plurality of stimulating electrodes are positioned on the paddle lead.

18. The method of claim 15, wherein implanting the neurostimulation system comprises implanting a neurostimulation system including a paddle lead and a cylindrical lead, wherein the plurality of stimulating electrodes are positioned on the paddle lead, and wherein the plurality of sensing electrodes are positioned on the cylindrical lead.

19. The method of claim 18, wherein implanting the neurostimulation system comprises positioning the cylindrical lead on one of a right tract of the tract of Lissauer and a left tract of the tract of Lissauer.

20. The method of claim 15, wherein implanting the neurostimulation system comprises implanting a neurostimulation system including a paddle lead, a first cylindrical lead, and a second cylindrical lead, wherein the plurality of stimulating electrodes are positioned on the paddle lead, wherein a first subset of the plurality of sensing electrodes are positioned on the first cylindrical lead, and wherein a second subset of the plurality of sensing electrodes are positioned on the second cylindrical lead.
